(19) United States
(12) Patent Application Publication
(10) Pub. No.: US 2006/0014970 A1
(43) Pub. Date: Jan. 19, 2006

(54) Method for the continuous production of epoxids from olefins and hydroperoxides on a suspended catalyst

(76) Inventors: Hans-Georg Goebbel, KALLSTADT (DE); Peter Bassler, Viernheim (DE); Joaquim Henrique Teles, Otterstadt (DE); Peter Rudolf, Ladenburg (DE); Georg Krug, Morlenbach (DE); Wolfgang Harder, Weinheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/531,438

(22) PCT Filed: Oct. 23, 2003
(86) PCT No.: PCT/EP03/11737

§ 371 Date: Apr. 14, 2005

(30) Foreign Application Priority Data
Oct. 23, 2002 (DE) 102 49 377.4

(51) Int. Cl. C07D 301/19

(52) U.S. Cl. 549/529

(57) ABSTRACT

Continuous process for the epoxidation of olefins by means of hydroperoxide, in which the epoxidation is carried out in a reactor in which at least one catalyst suspended in a liquid phase is present, wherein the liquid phase is passed through a device which has openings or channels and is installed in the reactor and the epoxide-containing liquid is separated off by means of crossflow filtration so that the suspended catalyst is retained in the reaction system.

The present invention relates to a continuous epoxidation process for converting olefins into epoxides in a reactor in which at least one catalyst suspended in a liquid phase and, if desired, additionally a gas phase are present, wherein the liquid phase and, if present, the gas phase are passed through a device having openings or channels in the reactor and the epoxide-containing liquid is separated off by means of a crossflow filtration so that the suspended catalyst is retained in the reaction system. The invention also relates to an apparatus for carrying out the process. Process and apparatus are preferably used in the epoxidation of propene by means of hydrogen peroxide to form propene oxide.

According to the prior art, the epoxidation of olefins by means of hydroperoxide can be carried out in one or more stages, with both batch processes and continuous processes being possible. The epoxidation is preferably also catalyzed, either in a heterogeneous or homogeneous phase. Processes are described, for example, in WO 00/07965.

Use of a fixed-bed reactor to carry out the heterogeneously catalyzed epoxidation is also known. For this purpose, specially prepared catalysts usually have to be produced. In such a use, the catalyst is preferably applied to support materials or processed to form specific shaped bodies. However, when the activity drops, which may occur after only relatively short periods of operation, the catalyst can often be removed from the fixed bed or regenerated only with some difficulty. This is usually associated with a shutdown of the entire plant, i.e. not only the epoxidation stage but also but also the following work-up stage. This leads to a low space-time yield, which is disadvantageous for an industrial process.

It is an object of the present invention to develop a process for the epoxidation of olefins by means of hydroperoxides, in which the catalyst can easily be replaced during the reaction without shutdown of the plant being necessary, while at the same time achieving a high space-time yield.

We have found that this object is achieved by a continuous process for the epoxidation of olefins, in which the epoxidation is carried out in a reactor in which at least one catalyst suspended in a liquid phase is present, wherein the liquid phase is passed through a device which has openings or channels and is installed in the reactor and the epoxide-containing liquid is separated off by means of crossflow filtration so that the suspended catalyst is retained in the reaction system.

If a gas phase is present, this too can be passed through the device which has openings or channels and is installed in the reactor.

The device having openings or channels through which the reaction medium is passed can comprise a bed, a knitted mesh or a packing element. Such devices are known from distillation and extraction technology.

However, for the purposes of the present invention, such devices in principle have a substantially smaller hydraulic diameter than the devices used as internals in distillation and extraction technology. In the novel process, this diameter is preferably smaller by a factor of from 2 to 10. The hydraulic diameter of the device used as internal in the reactor in the process of the present invention is preferably from 0.5 to 20 mm.

The hydraulic diameter is a characteristic quantity for the description of the equivalent diameter of non-circular openings or channel structures.

In the context of the present invention, the term “hydraulic diameter” relates to the ratio of four times the cross-section of the opening and the circumference of the opening. In case a channel structure having a cross-section in the shape of an isosceles triangle is concerned, the term “hydraulic diameter” relates to the quantity 2bk/(b+2s) wherein b is the length of the basis, k is the height and s is the length of the lateral side of the triangle.

Packing elements which offer the advantage of a low pressure drop are, for example, woven wire mesh packings. Apart from woven mesh packings, it is also possible to use packings comprising other woven, knitted or felted liquid-permeable materials.

Further suitable packings or packing elements which can be used are flat metal sheets, preferably without perforation or other relatively large openings. Examples are commercial types such as B1 from Montz or Mellapak from Sulzer.

Packings made of expanded metal, for example BSH packing from Montz, are also advantageous. Here too, openings which are, for instance, in the form of perforations have to be kept appropriately small. The decisive factor determining the suitability of packing for the purposes of the present invention is not its geometry but the widths of openings or channels in the packing which allow flow to occur.

To suspend the solid particles in the reactor, mechanical energy is introduced into the reactor, preferably by means of stirrers, nozzles or rising gas bubbles. The installation of the abovementioned devices in the reactor produces an increases difference in the motion of the catalyst particles relative to the liquid phase in the reaction section, since the particles are held back more strongly than the surrounding liquid in the narrow openings and channels of these devices. This increased relative velocity improves mass transfer between liquid and suspended particles, which is important for achieving a high space-time yield.

The use of catalyst particles having particle sizes in the range from 1 to 10 mm for suspension catalysts is also known. Although particles of this size have the desired relative velocity relative to the surrounding liquid, their low surface area per unit volume limits turnover. The two effects frequently cancel out one another, so that the problem of increasing mass transport is not solved in the final analysis.

In contrast thereto, the catalyst particles used in the process of the present invention preferably have a mean particle size of from 0.0001 to 2 mm, more preferably from 0.0001 to 1 mm, particularly preferably from 0.005 to 0.1 mm. Particles of this mean particle size surprisingly enable the relative velocity and mass transport to be increased further.

In the novel process, the high relative velocity which can be achieved is also extremely advantageous compared to processes in which reactors without the abovementioned internals are used. Increasing the introduction of mechanical energy above that required for achieving suspension leads to no appreciable improvement in mass transfer between the liquid and the suspended solid particles in suspension reactors without internals, since the relative velocity which can be achieved is only insignificantly higher than the sedimentation velocity.

When the internals in the reactor are combined with catalyst particles in the particle size range indicated, high relative velocities of the liquid phase relative to the catalyst particles and thus advantageous mass transport are achieved. The novel process is therefore superior to processes in which no internals are used in the reactor or catalyst particles having a greater diameter are used.

The process can be carried out in various continuously operated types of reactor, e.g. jet nozzle reactors, bubble columns or shell-and-tube reactors. It is not necessary for the internals to fill the entire reactor.

Particularly preferred embodiments of the reactor are bubble columns or shell-and-tube reactors.

A very particularly preferred reactor is a heatable and coolable shell-and-tube reactor in which the internals are accommodated in the individual tubes. Such a reactor has the advantage that the energy required for activation of the reaction can be readily introduced or the heat of reaction evolved can be readily removed.

Preference is given to the reactor being arranged vertically and the reaction mixture flowing through it from the bottom upward.

In the process of the present invention, the epoxidation is carried out in a reactor having one of the above-described internals in the presence of one or more suspension catalysts at a pressure of from 1 to 100 bar, preferably from 1 to 60 bar, particularly preferably from 1 to 50 bar. The reaction temperature is in the range from 20 to 100° C., preferably from 30 to 80° C., particularly preferably from 40 to 70° C.

The process is simple to carry out. The above-described device, preferably woven mesh packing or sheet metal packing, is installed in the reactor. The reaction mixture comprising olefin, hydroperoxide and suspension catalyst is then circulated at high velocity through the reactor by means of a pump. The throughput per unit cross-sectional area (empty tube velocity) of the liquid phase is preferably from 50 to 300 m³/m² h, in particular in the range from 100 to 250 m³/m² h.

The suspended catalyst material is introduced into the reactor with the aid of customary techniques. Retention of the suspension catalyst in the reaction system while the epoxide-containing liquid phase is separated off is achieved by the use of crossflow filtration.

Membranes suitable for the crossflow filtration are specifically treated aluminum oxide or sintered metal membranes having pore diameters of from 50 to 500 nm, preferably from 50 to 100 nm, as are marketed by, for example, Membraflow. The membrane modules, in general multichannel modules, are installed in the reaction circuit in such a way that the flow velocity in the individual channels is from 1 to 6 m/s, preferably from 2 to 4 m/s, and no deposit can settle on the membrane surfaces as a result. The permeate stream, i.e. the epoxide-containing liquid stream which passes through the membrane, is taken off perpendicular to the main flow direction. The amount is regulated via the prevailing trans-membrane pressure. A trans-membrane pressure in the range from 0.2 to 2 bar, preferably from 0.3 to 1 bar, is desirable. The trans-membrane pressure is defined as the difference between the mean pressure on the feed or retentate side and the pressure on the permeate side.

The epoxide-containing liquid is obtained as permeate and can be passed to work-up.

If the activity of the catalyst drops to such an extent that the process proceeds only unsatisfactorily, it can be conveniently separated off the system, replaced or regenerated. Preference is given to part of the catalyst suspension being discharged from the system during the reaction and being replaced by fresh catalyst suspension. The deactivated catalyst can then be regenerated externally. Interruption of the epoxidation stage or the work-up stage of the epoxide-containing liquid is thus not necessary, which is extremely advantageous.

In the process, the epoxide-containing solution is replaced by starting materials and solvent in the amount corresponding to that in which the solution is taken off. This makes a continuously operated process possible, which is extremely useful for industrial implementation.

The starting materials known from the prior art can be used for the epoxide synthesis in the process of the present invention.

Preference is given to using organic compounds which have at least one C—C double bond. Examples of such organic compounds having at least one C—C double bond are the following alkenes:

ethene, propene, 1-butene, 2-butene, isobutene, butadiene, pentenes, piperylene, hexenes, hexadienes, heptenes, octenes, diisobutene, trimethylpentene, nonenes, dodecene, tridecene, tetradecenes to eicosenes, tripropene and tetrapropene, polybutadienes, polyisobutenes, isoprenes, terpenes, geraniol, linalool, linalyl acetate, methylenecyclopropane, cyclopentene, cyclohexene, norbornen, cycloheptene, vinylcyclohexane, vinyloxirane, vinylcyclohexene, styrene, cyclooctene, cyclooctadiene, vinylnorbomene, indene, tetrahydroindene, methylstyrene, dicyclopentadiene, divinylbenzene, cyclododecene, cyclododecatriene, stilbene, diphenylbutadiene, vitamin A, beta-carotene, vinylidene fluoride, allyl halides, crotyl chloride, methallyl chloride, dichlorobutene, allyl alcohol, methallyl alcohol, butenols, butenediols, cyclopentenediols, pentenols, octadienols, tridecenols, unsaturated steroids, ethoxyethene, isoeugenol, anethole, unsaturated carboxylic acids such as acrylic acid, methacrylic acid, crotonic acid, maleic acid, vinylacetic acid, unsaturated fatty acids such as oleic acid, linoleic acid, palmitic acid, naturally occurring fats and oils.

Particular preference is given to using alkenes which contain from 2 to 8 carbon atoms, e.g. ethene, propene and butene.

Very particular preference is given to using propene.

It is also possible to use “chemical grade” propene. In this case, propene is present together with propane in a volume ratio of propene to propane of from about 97:3 to 95:5.

As hydroperoxides, it is possible to use the known hydroperoxides which are suitable for the reaction of the organic compound. Examples of such hydroperoxides are tert-butyl hydroperoxide or ethylbenzene hydroperoxide. Hydrogen peroxide is preferably used as hydroperoxide for the epoxide synthesis, preferably as an aqueous hydrogen peroxide solution.

As heterogeneous catalysts, use is made of ones which comprise a porous oxidic material, e.g. a zeolite. Preference is given to using catalysts which comprise a titanium-, germanium-, tellurium-, vanadium-, chromium-, niobium- or zirconium-containing zeolite as porous oxidic material.

Specific examples are titanium-, germanium-, tellurium-, vanadium-, chromium-, niobium-, zirconium-containing zeolites having a pentasil zeolite structure, in particular the types which can be assigned X-ray crystallographically to the ABW, ACO, AEI, AEL, AEN, AET, AFG, AFI, AFN, AFO, AFR, AFS, ATF, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CFI, CLO, CON, CZP, DAC, DDR, DFO, DFF, DOH, DON, EAB, EDI, EMT, EPI, ERI, ESV, EUO, FAU, FER, GIS, GME, GOO, HEU, IFR, ISV, IFE, JBW, KFI, LAU, LEV, LIO, LOS, LOV, LTA, LTL, LTN, MAZ, MEI, MEL, MEP, MER, MFI, MFS, MON, MOR, MSO, MTF, MTN, MTT, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG, ZON structure or to mixed structures of two or more of the abovementioned structures. The use of titanium-containing zeolites having the ITQ-4, SSZ-24, TTM-1, UTD-1, CIT-1 or CIT-5 structure is also conceivable in the process of the present invention. Further titanium-containing zeolites which may be mentioned are those having the ZSM-48 or ZSM-12 structure.

Particular preference is given to Ti zeolites having the MFI or MEL structure or the MFI/MEL mixed structure. Very particular preference is given to the titanium-containing zeolite catalysts which are generally referred to as “TS-1”, “TS-2” and “TS-3”, and also Ti zeolites having a lattice structure isomorphous with β-zeolite.

The use of a heterogeneous catalyst comprising the titanium-containing silicalite TS-1 is very advantageous.

It is possible, inter alia, to use the porous oxidic material itself as catalyst. However, it is also possible to use a shaped body comprising the porous oxidic material as catalyst. To produce the shaped body from the porous oxidic material, it is possible to employ all processes known from the prior art.

In these processes, noble metals can be applied in the form of suitable noble metal components, for example in the form of water-soluble salts, to the catalyst material before, during or after one or more shaping steps. This method is preferably employed for producing oxidation catalysts based on titanium silicates or vanadium silicates having a zeolite structure, and makes it possible to obtain catalysts having a content of from 0.01 to 30% by weight of one or more noble metals from the group consisting of ruthenium, rhodium, palladium, osmium, iridium, platinum, rhenium, gold and silver. Such catalysts are described, for example, in DE-A 196 23 609.6.

Of course, the shaped bodies can be subjected to finishing treatment. All methods of comminution, for example milling, splitting or crushing of the shaped bodies, and also further chemical treatments as described by way of example above are conceivable.

When using a shaped body or a plurality thereof as catalyst, this can, after it has been deactivated, be regenerated in the process of the present invention by a method in which regeneration is achieved by targeted burning-off of the deposits responsible for deactivation. This is preferably carried out in an inert gas atmosphere containing precisely defined amounts of oxygen-donating substances. This regeneration process is described in DE-A 197 23 949.8. It is also possible to use the regeneration processes cited there in the discussion of the prior art.

As solvents, preference is given to using all solvents which completely or at least partly dissolve the starting materials used in the epoxide synthesis. For example, it is possible to use water; alcohols, preferably lower alcohols, more preferably alcohols having less than 6 carbon atoms, for example methanol, ethanol, propanols, butanols, pentanols, diols or polyols, preferably those having less than 6 carbon atoms; ethers such as diethyl ether, tetrahydrofuran, dioxane, 1,2-diethoxyethane, 2-methoxyethanol; esters such as methyl acetate or butyrolactone; amides such as dimethylformamide, dimethylacetamide, N-methylpyrrolidone; ketones such as acetone; nitrites such as acetonitrile; sulfoxides such as dimethyl sulfoxide; aliphatic, cycloaliphatic and aromatic hydrocarbons, or mixtures of two or more of the abovementioned compounds.

Preference is given to using alcohols. Here, the use of methanol as solvent is particularly preferred.

In the reaction of the olefin with the hydroperoxide, it is also possible for further compounds which are customarily used in epoxidation reactions to be present. Such compounds are, for example, buffers by means of which the pH range favorable for the respective epoxidation can be set and the activity of the catalyst can be regulated.

The invention further provides an apparatus for carrying out a continuous process for the epoxidation of olefins by means of hydroperoxide as is described above, comprising a reactor in which the epoxidation is carried out, a crossflow filter for separating off epoxide-containing solution so that the catalyst is retained in the reactor and a container for the catalyst suspension.

In particular, the apparatus for carrying out a continuous process for the epoxidation of olefins comprises a reactor having internals selected from the group consisting of beds, knitted meshes or packing elements and having a hydraulic diameter of from 0.5 to 20 mm, a catalyst having a mean particle size of from 0.0001 to 2 mm suspended in a liquid, a crossflow filter and a container for the catalyst suspension.

In a particularly preferred embodiment of the apparatus for carrying out the process, the reactor is a bubble column or a shell-and-tube reactor. In a very particularly preferred embodiment, the reactor is a shell-and-tube reactor which makes heat removal possible.

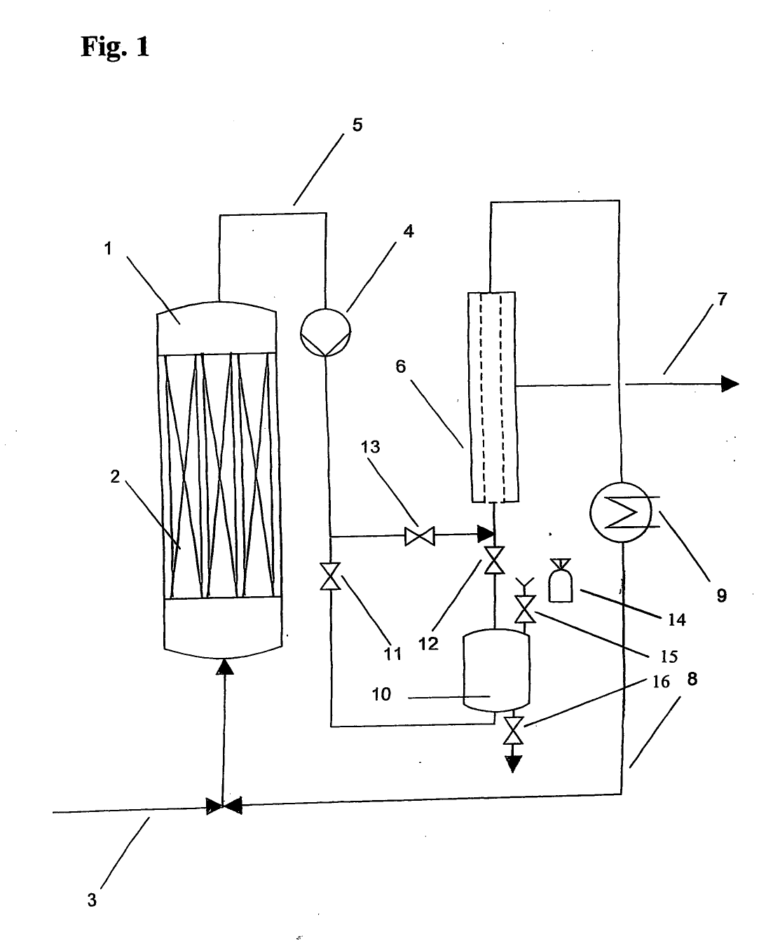

A reactor for the epoxidation of olefins will now be described by way of example with the aid of FIG. 1. In such a reactor, preference is given to reacting propene with hydrogen peroxide as epoxidizing agent in methanol as solvent using a suspended TS-1 catalyst and, if appropriate, buffer additives for controlling the reactivity of the catalyst and the pH to give propene oxide.

FIG. 1 shows, by way of example, the experimental structure of a continuously operated reactor 1, e.g. a bubble column or particularly preferably a heatable and coolable shell-and-tube reactor, which is provided with heatable packings 2 and which is supplied via the lines 3 with a liquid mixture comprising the olefin, hydrogen peroxide, the solvent and, if appropriate, buffer additives. The pump 4 maintains the circulation and thus keeps the catalyst in suspension. After leaving the reactor 1, the reaction solution is conveyed via line 5 to the crossflow filter 6. The permeate is taken off perpendicular to the main flow direction and is passed via the line 7 to the work-up stage of the plant.

Since the catalyst cannot pass the crossflow filter, it remains suspended in the reactor system and is conveyed via line 8 and, if appropriate, the heat exchanger 9 to the reactor 1, thus closing the catalyst circuit.

Introduction or discharge of the catalyst is carried out, for example, via a container 10 which can be incorporated in a specific fashion in the reaction circuit. To introduce catalysts, a particular amount of catalyst is, for example, placed in the container and the latter is filled with solvent. The valves 11 and 12 are subsequently opened and the valve 13 is closed. In this state, all the reaction medium flows through the container 10 and the catalyst is carried into the system.

A similar procedure is used to discharge catalyst. The container 10 is filled, for example, with methanol and the valves 11 and 12 are subsequently opened and the valve 13 is closed. The reaction medium once again flows through the reactor. After the catalyst concentrations in the reactor and the container have become equal, the valves 11 and 12 are closed and the valve 13 is opened. The container 10 is now isolated from the reaction medium and contains an aliquot of catalyst. This can then be separated from the solution in a further step and possibly be regenerated externally. After regeneration, it can be fed back into the system as described above.

Via valve 15, catalyst material can be introduced in container 10.

### LIST OF REFERENCE NUMERALS FOR FIG. 1

1 Reactor (bubble column, shell-and-tube reactor)
2 Packings
3 Feed line
4 Pump
5 Line
6 Crossflow filter
7 Line for the permeate
8 Line
9 Heat exchanger
10 Container for catalyst suspension
11 Valve
12 Valve
13 Valve
14 Catalyst material
15 Valve
16 Valve

1-7. (canceled)

8. A continuous process for the epoxidation of olefins by means of hydroperoxide, wherein the epoxidation is carried out in a reactor in which at least one catalyst suspended in a liquid phase is present in the form of particles having a mean particle size of from 0.0001 to 2 mm, and wherein the liquid phase is passed through a device which has openings or channels and is installed in the reactor and the catalyst is retained in the reaction system by means of crossflow filtration when the epoxide containing liquid is separated off, wherein the crossflow filtration is carried out using membrane modules installed in the reaction circuit in such a way that the flow velocity in the individual channels is from 1 to 6 m/s and wherein catalyst suspension is taken from or fed into the reactor during the epoxidation.

9. A process as claimed in claim 8, wherein a gas phase which is present in the reactor is also passed through the device which has openings or channels and is installed in the reactor.

10. A process as claimed in claim 8, wherein the hydraulic diameter of the device installed in the reactor is from 0.5 to 20 mm.

11. A process as claimed in claim 9, wherein the hydraulic diameter of the device installed in the reactor is from 0.5 to 20 mm.

12. A process as claimed in claim 8, wherein the device installed in the reactor is a bed, a knitted mesh or a packing element.

13. A process as claimed in claim 9, wherein the device installed in the reactor is a bed, a knitted mesh or a packing element.

14. A process as claimed in claim 10, wherein the device installed in the reactor is a bed, a knitted mesh or a packing element.

15. A process as claimed in claim 8, wherein the reactor is a jet nozzle reactor, a bubble column or a shell-and-tube reactor.

16. A process as claimed in claim 9, wherein the reactor is a jet nozzle reactor, a bubble column or a shell-and-tube reactor.

17. A process as claimed in claim 10, wherein the reactor is a jet nozzle reactor, a bubble column or a shell-and-tube reactor.

18. A process as claimed in claim 8, wherein the epoxidation is carried out at a temperature of from 20 to 100° C. and a pressure of from 1 to 100 bar.

19. A process as claimed in claim 9, wherein the epoxidation is carried out at a temperature of from 20 to 100° C. and a pressure of from 1 to 100 bar.

20. A process as claimed in claim 10, wherein the epoxidation is carried out at a temperature of from 20 to 100° C. and a pressure of from 1 to 100 bar.

21. A process as claimed in claim 8, wherein propene is epoxidized by means of hydrogen peroxide over a titanium-containing zeolite.

22. A process as claimed in claim 9, wherein propene is epoxidized by means of hydrogen peroxide over a titanium-containing zeolite.

23. A process as claimed in claim 10, wherein propene is epoxidized by means of hydrogen peroxide over a titanium-containing zeolite.

24. A continuous process for the epoxidation of olefins by means of hydroperoxide, wherein the epoxidation is carried out in a reactor in which at least one catalyst suspended in a liquid phase is present in the form of particles having a mean particle size of from 0.0001 to 2 mm, and wherein the liquid phase is passed through a device which has openings or channels and is installed in the reactor and the catalyst is retained in the reaction system by means of crossflow filtration when the epoxide containing liquid is separated off, wherein the crossflow filtration is carried out using membrane modules installed in the reaction circuit in such a way that the flow velocity in the individual channels is from 1 to 6 m/s, wherein catalyst suspension is taken from or fed into the reactor during the epoxidation and wherein a gas phase which is present in the reactor is also passed through the device which has openings or channels and is installed in the reactor.

25. A continuous process for the epoxidation of olefins by means of hydroperoxide, wherein the epoxidation is carried out in a reactor in which at least one catalyst suspended in a liquid phase is present in the form of particles having a mean particle size of from 0.0001 to 2 mm, and wherein the liquid phase is passed through a device which has openings or channels and is installed in the reactor and the catalyst is retained in the reaction system by means of crossflow filtration when the epoxide containing liquid is separated off, wherein the crossflow filtration is carried out using membrane modules installed in the reaction circuit in such a way that the flow velocity in the individual channels is from 1 to 6 m/s, wherein catalyst suspension is taken from or fed into the reactor during the epoxidation and wherein propene is epoxidized by means of hydrogen peroxide over a titanium-containing zeolite.

26. A continuous process for the epoxidation of olefins by means of hydroperoxide, wherein the epoxidation is carried out in a reactor in which at least one catalyst suspended in a liquid phase is present in the form of particles having a mean particle size of from 0.0001 to 2 mm, and wherein the liquid phase is passed through a device which has openings or channels and is installed in the reactor and the catalyst is retained in the reaction system by means of crossflow filtration when the epoxide containing liquid is separated off, wherein the crossflow filtration is carried out using membrane modules installed in the reaction circuit in such a way that the flow velocity in the individual channels is from 1 to 6 m/s, wherein catalyst suspension is taken from or fed into the reactor during the epoxidation, wherein a gas phase which is present in the reactor is also passed through the device which has openings or channels and is installed in the reactor and wherein the hydraulic diameter of the device installed in the reactor is from 0.5 to 20 mm.

27. A continuous process for the epoxidation of olefins by means of hydroperoxide, wherein the epoxidation is carried out in a reactor in which at least one catalyst suspended in a liquid phase is present in the form of particles having a mean particle size of from 0.0001 to 2 mm, and wherein the liquid phase is passed through a device which has openings or channels and is installed in the reactor and the catalyst is retained in the reaction system by means of crossflow filtration when the epoxide containing liquid is separated off, wherein the crossflow filtration is carried out using membrane modules installed in the reaction circuit in such a way that the flow velocity in the individual channels is from 1 to 6 m/s, wherein catalyst suspension is taken from or fed into the reactor during the epoxidation, wherein the hydraulic diameter of the device installed in the reactor is from 0.5 to 20 mm and wherein propene is epoxidized by means of hydrogen peroxide over a titanium-containing zeolite.
